(19) United States
(12) Patent Application Publication
(10) Pub. No.: US 2025/0164290 A1
(43) Pub. Date: May 22, 2025

(54) OPTICAL FIBER ANCHOR FOR DISTRIBUTED SENSING IN BRITTLE MEDIUMS

(71) Applicant: THE CURATORS OF THE UNIVERSITY OF MISSOURI, Columbia, MO (US)

(72) Inventors: Taghi Sherizadeh, Rolla, MO (US); Samuel Nowak, Houston, TX (US); Mina Esmaeelpour, Rolla, MO (US)

(73) Assignee: The Curators of the University of Missouri, Columbia, MO (US)

(21) Appl. No.: 18/840,657

(22) PCT Filed: Feb. 28, 2022
(86) PCT No.: PCT/US2022/018157

§ 371 Date: Aug. 22, 2024

(51) Int. Cl. G01D 5/353; G02B 6/44

(52) U.S. Cl. CPC G01D 5/353; G02B 6/443

(57) ABSTRACT

A fiber optic cable system configured for distributed sensing in a brittle installation medium, the fiber optic cable system comprising a fiber optic cable and a plurality of anchors. The fiber optic cable includes a fiber core, a buffer layer, a strength member, and a jacket. These components are secured together at a number of anchoring locations along the fiber optic cable so that they are bonded together at the anchoring locations and un-bonded from each other between the anchoring locations. The anchors are attached to the fiber optic cable at the anchoring locations and are configured to engage the brittle installation medium to affix the fiber optic cable to the brittle installation medium at the anchoring locations.

### GOVERNMENT INTERESTS

This invention was made with government support under Contract No. 75D30120C09233 awarded by the National Institute for Occupational Safety and Health. The government has certain rights in the invention.

### BACKGROUND

Fiber optic cables typically include a fiber core for data transmission and sensing, a buffer layer, a strength member of aramid yarn or other loose material, and an outer jacket. Fiber optic cables with loose interior layers are not very suitable for distributed strain measurements because the loose interior layers can slip within the jacket, causing erroneous strain measurements. Conversely, fiber optic cables with tight interior layers are commercially available for strain sensing purposes, but when used in installation media such as in cement within a borehole or in sand within a trench, the fiber optic cable can slip relative to the installation media when strained, causing erroneous measurements.

The spatial resolution of a fiber optic distributed sensing system is a function of light pulse duration and maximum sensing range. If the length of the portion of fiber optic cable experiencing strain is less than the spatial resolution of the distributed sensing system being used, a lower-than-expected measured strain value is produced, or in extreme cases, no measurement is possible.

### SUMMARY

The present invention solves the above problems and other problems and provides a distinct advance in the art of distributed sensing.

A fiber optic cable system constructed in accordance with an embodiment of the invention comprises a fiber optic cable and a number of anchors.

The fiber optic cable includes a number of fiber cores, a number of buffer layers, a strength member, and a jacket. The fiber optic cable is resiliently flexible and configured to be communicatively connected to (and part of) a distributed sensing system.

The fiber cores are configured to carry an optical signal and may be made of glass, plastic, or any other suitable material. Although dual-core construction is shown, other multi-core or single core construction may be used.

The buffer layers protect and provide mechanical support to the fiber cores. The buffer layers are coatings of polymer or similar material forming small conduits encircling the fiber cores.

The strength member encircles the fiber cores and the buffer layers and may include yarn (e.g., Aramid yarn), fiberglass epoxy rods, synthetic fiber (e.g., Nylon), steel wire, or the like. The strength member provides strength and durability to the fiber optic cable, while being at least partially flexible.

The jacket encircles the fiber cores, the buffer layers, and the strength member. The jacket is made of a plenum plastic material or other suitable material for providing environmental protection to the fiber cores, the buffer layers, and the strength member.

The fiber cores, the buffer layers, the strength member, and the jacket are secured together at a number of anchoring locations spaced an interval length apart from each other so that the fiber cores, the buffer layers, the strength member, and the jacket are bonded together at the anchoring locations and unbonded from each other between the anchoring locations.

Each anchor may include opposing ends and structure configured to engage the brittle installation medium. In one embodiment, the structure may include a number of ribs defining an infiltration space.

The ribs include four orthogonally-spaced ribs extending between and merging together at the opposing ends. The ribs protrude radially from a longitudinal axis of the anchor.

The infiltration space receives brittle installation medium when the brittle installation medium is in an at least semi-liquid state (e.g., wet cement). To that end, the infiltration space is a large central void between the ribs to ensure the brittle installation medium thoroughly fills around the ribs.

Fabrication of the fiber optic cable and constructing the fiber optic cable system includes securing the fiber cores, the buffer layers, the strength member, and the jacket together at a number of anchoring locations spaced an interval length apart from each other so that these components are bonded together at the anchoring locations and unbonded from each other between the anchoring locations. This may include melting the jacket at the anchoring locations via a heating element such as a heat shrink gun.

The jacket may then be compressed around the fiber cores at the anchor locations. This may be achieved via shrinking of the jacket as a result of heating or melting the jacket. Alternatively or additionally, the jacket may be crimped, thus flattening the jacket and potentially the strength member and the buffer layers.

The jacket may then be cooled, resulting in the jacket being coupled to the fiber cores via the buffer layers and strength member. In other words, the various layers and components of the fiber optic cable are thereby coupled together and unable to slip or move relative to each other at the plurality of anchoring locations. Cooling may be achieved actively via a cooling system or cooling mechanism or may be achieved passively via ambient temperatures.

The above steps may be performed at each of the plurality of anchoring locations simultaneously or nearly simultaneously, or the above steps may be performed at each of the plurality of anchoring locations in succession, or in some combination thereof.

The jacket and fiber cores are not coupled together between the anchoring locations so that the fiber cores can move relative to the jacket, particularly when the fiber optic cable is under shear or tension between nearby anchoring locations.

The anchors are then positioned along the fiber optic cable at the anchoring locations so the coupled portions of the fiber optic cable are at least partially positioned in the infiltration spaces defined by the anchors. The anchors are then attached to the fiber optic cable via cable ties or similar fasteners.

The above-described fiber optic cable system and method provides several advantages. For example, the fiber optic cable is anchored at anchoring locations spaced apart at known intervals, preferably equal to or greater than the spatial resolution of the sensing system being used. The fiber cores are un-bonded from the installation medium between the anchoring locations, which provides significantly improved monitoring of shear and tension deformations within the installation medium. It also provides crack detection with a spatial resolution akin to that of the sensing system being used.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is a partial elevation view of a fiber optic cable system constructed in accordance with an embodiment of the invention;

FIG. 2a is an enlarged partial elevation view of a portion of the fiber optic cable system of FIG. 1;

FIG. 2b is an enlarged partial elevation view of a portion of the fiber optic cable system of FIG. 1;

FIG. 3 is a partial cutaway perspective view of a fiber optic cable of the fiber optic cable system of FIG. 1;

FIG. 4 is a partial cutaway elevation view of a fiber optic cable system constructed in accordance with an embodiment of the invention;

FIG. 5 is a partial cutaway elevation view of a fiber optic cable system constructed in accordance with an embodiment of the invention; and

FIG. 6 is a flow diagram of certain steps of a method of fabricating a fiber optic cable system in accordance with an embodiment of the invention.

The drawing figures do not limit the present invention to the specific embodiments disclosed and described herein. The drawings are not necessarily to scale, emphasis instead being placed upon clearly illustrating the principles of the invention.

### DETAILED DESCRIPTION OF THE EMBODIMENTS

The following detailed description of the invention references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense. The scope of the present invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

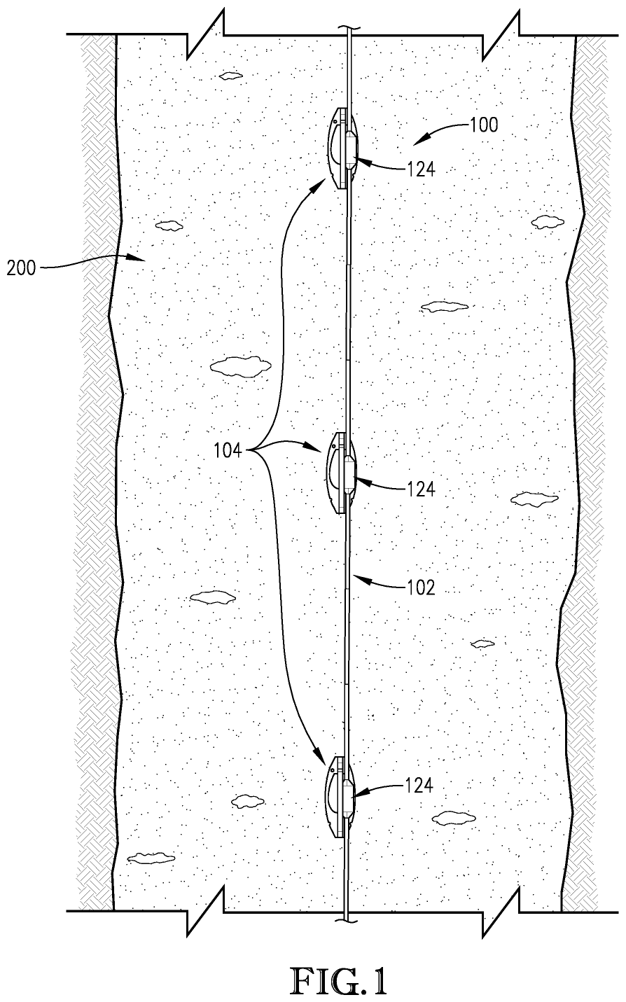

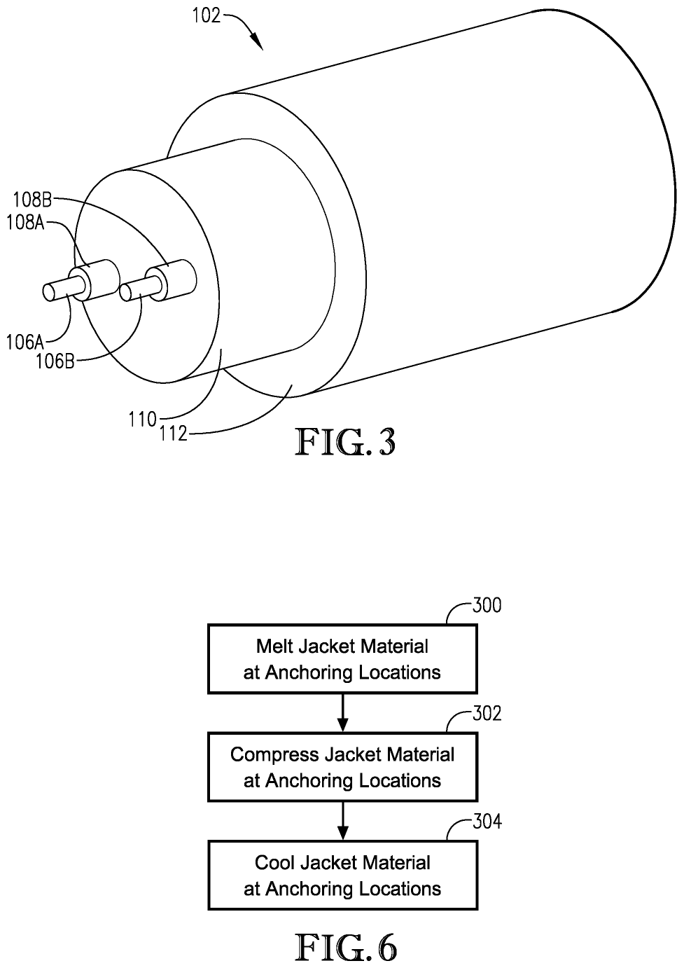

Turning now to the drawing figures, and particularly FIGS. 1-5, a fiber optic cable system 100 constructed in accordance with an embodiment of the invention is illustrated. The fiber optic cable system 100 is configured for distributed sensing in a brittle installation medium, such as brittle installation medium 200, and broadly comprises a fiber optic cable 102 and a plurality of anchors 104.

The fiber optic cable 102 may include fiber cores 106A, B, buffer layers 108A, B, a strength member 110, and a jacket 112. The fiber optic cable 102 may be resiliently flexible and configured to be communicatively connected to (and part of) a distributed sensing system.

The fiber cores 106A, B may be configured to carry an optical signal and may be made of glass, plastic, or any other suitable material. Although dual-core construction is shown, other multi-core or single core construction may be used.

The buffer layers 108A, B protect and provide mechanical support to the fiber cores 106A,B. The buffer layers 108A,B may be coatings of polymer or similar material forming small conduits encircling the fiber cores 106A, B.

The strength member 110 may encircle the fiber cores 106A, B and the buffer layers 108A, B and may include yarn (e.g., Aramid yarn), fiberglass epoxy rods, synthetic fiber (e.g., Nylon), steel wire, or the like. The strength member 110 provides strength and durability to the fiber optic cable 102, while being at least partially flexible.

The jacket 112 may encircle the fiber cores 106A,B, the buffer layers 108A, B, and the strength member 110. The jacket 112 may be made of a plenum plastic material or any other suitable material for providing environmental protection to the fiber cores 106A, B, the buffer layers 108A, B, and the strength member 110. To that end, the jacket 112 may be waterproof or water resistant, UV resistant, and have abrasion and crack resistance.

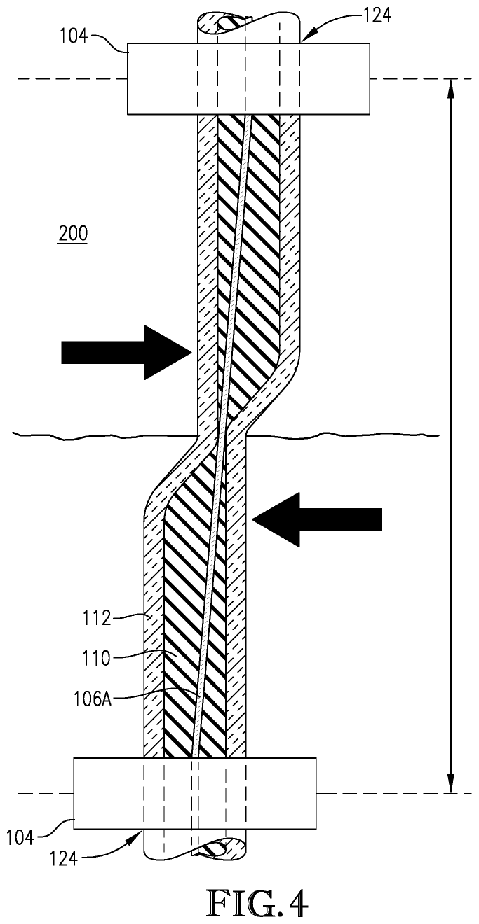

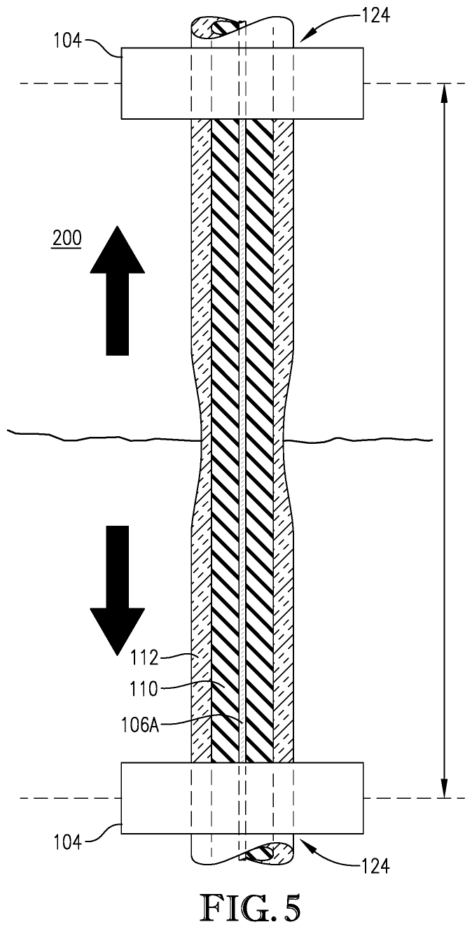

As discussed in more detail below, the fiber cores 106A,B, the buffer layers 108A, B, the strength member 110, and the jacket 112 may be secured together at a plurality of anchoring locations 124 spaced an interval length apart from each other (see spacing in FIGS. 4 and 5 represented by vertical dimensional arrows) so that the fiber cores 106A, B, the buffer layers 108A, B, the strength member 110, and the jacket 112 are bonded together at the plurality of anchoring locations 124 and unbonded from each other between the plurality of anchoring locations 124.

The plurality of anchors 104 may be substantially similar and thus only one anchor 104 will be described in detail. The anchor 104 may include opposing ends 114A, B and structure configured to engage the brittle installation medium 200. In one embodiment, the structure may include a plurality of ribs 116A-D defining an infiltration space 118. The anchor 104 may be made of any suitable rigid or semi-rigid material. Anchors 104 are shown as blocks in FIGS. 4 and 5 for simplicity.

In one embodiment, the ribs 116A-D may include four orthogonally-spaced ribs 116A-D extending between and merging together at the opposing ends 114A,B. The ribs 116A-D may protrude radially from a longitudinal axis of the anchor 104. The ribs 116A-D are shown having an arcuate bow, but other configurations and shapes such as cantilever fins may be used.

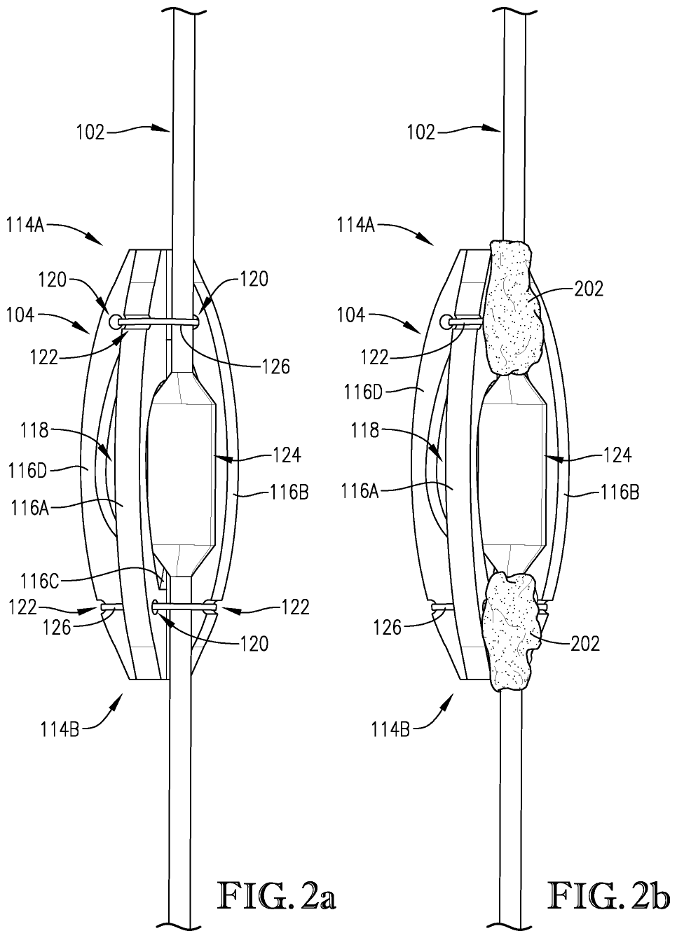

The ribs 116A-D may define a plurality of fastener apertures 120 and fastener guides 122 for receiving cinching fasteners such as cable ties, as best seen in FIG. 2a. In one embodiment, a first two opposing ribs 116A,C include fastener apertures near one end and fastener guides near their other end. A second two opposing ribs 116B, D include fastener apertures near one end in alignment with the fastener guides of the first two opposing ribs and fastener guides near the other end in alignment with the fastener apertures of the first two opposing ribs. In this way, a cinching fastener can be secured in alternating two fastener apertures and fastener guides near ends of the ribs 116A-D.

The infiltration space 118 may receive some of the brittle installation medium 200 when the brittle installation medium 200 is in an at least semi-liquid state (e.g., wet cement). To that end, the infiltration space 118 may be a large central void between the plurality of ribs 116 to ensure the brittle installation medium 200 thoroughly fills around the ribs 116. The anchor 104 may further define smaller spaces, channels, tunnels, openings, porous features, jagged or irregular geometry, guides, protrusions, or the like to ensure the anchor 104 is embedded in and mechanically fixed to the brittle installation medium 200.

Turning to FIG. 6, fabricating the fiber optic cable 102 and constructing the fiber optic cable system 100 will now be described in more detail. Significantly, the fiber cores 106A, B, the buffer layers 108A, B, the strength member 110, and the jacket 112 may be secured together at a plurality of anchoring locations 124 spaced an interval length apart from each other so that the fiber cores 106A,B, the buffer layers 108A,B, the strength member 110, and the jacket 112 are bonded together at the plurality of anchoring locations 124 and unbonded from each other between the plurality of anchoring locations 124.

In one embodiment, securing the fiber cores 106A,B, the buffer layers 108A, B, the strength member 110, and the jacket 112 together at the plurality of anchoring locations 124 may include melting the jacket 112 at the plurality of anchoring locations, as shown in block 300. This may be achieved via a heating element such as a heat shrink gun.

The jacket 112 may then be compressed around the fiber cores 106A, B at the plurality of anchor locations, as shown in block 302. This may be achieved via shrinking of the jacket 112 as a result of heating or melting the jacket 112. Alternatively or additionally, the jacket 112 may be crimped, thus flattening the jacket 112 and potentially the strength member 110 and the buffer layers 108A,B.

The jacket 112 may then be cooled, resulting in the jacket 112 being coupled to the fiber cores 106A,B via the buffer layers 108A,B and strength member 110, as shown in block 304. In other words, the various layers and components of the fiber optic cable 102 are thereby coupled together and unable to slip or move relative to each other at the plurality of anchoring locations. Cooling may be achieved actively via a cooling system or cooling mechanism or may be achieved passively via ambient temperatures.

The jacket 112 and fiber cores 106A, B may also be coupled together via other means. For example, adhesives, fasteners, interlocking components, and the like, or a combination thereof, may be used.

The above steps may be performed at each of the plurality of anchoring locations simultaneously or nearly simultaneously, or the above steps may be performed at each of the plurality of anchoring locations in succession, or in some combination thereof.

The jacket 112 and fiber cores 106A,B may not be coupled together between the plurality of anchoring locations so that the fiber cores 106A,B can move relative to the jacket, particularly when the fiber optic cable 102 is under shear or tension between nearby anchoring locations.

The anchors 104 may then be positioned along the fiber optic cable 102 at the plurality of anchoring locations so the coupled portions of the fiber optic cable 102 are at least partially positioned in the infiltration spaces defined by the anchors 104. In one embodiment, the fiber optic cable 102 may be threaded through the anchors 104.

After the anchors 104 are positioned, they may be attached to the fiber optic cable 102 via cable ties 126 or similar fasteners. In one embodiment, cable ties 126 may be threaded through the plurality of fastener apertures and aligned in the plurality of fastener guides to draw the anchors 104 close to the fiber optic cable 102 and to retain the anchors 104 in place.

Cementitious epoxy resin 202 or similar binding material may then be applied to the anchors 104 and the fiber optic cable 102 to bind anchors 104 to the fiber optic cable 102, as best seen in FIG. 2b. In one embodiment, the binding material may be applied near ends of the ribs 116 near the cable ties 126, where the fiber optic cable 102 is most rigidly constrained to the anchors 104. The cable ties 126 may give the binding material additional binding interface, while the binding material also prevents the cable ties 126 from loosening.

The above-described fiber optic cable system 100 and method provides several advantages. For example, the fiber optic cable 102 may be anchored at anchoring locations spaced apart at known intervals, preferably equal to or greater than the spatial resolution of the sensing system being used. The fiber cores 106A, B may be un-bonded from the installation medium 200 between the anchoring locations, while being protected from harsh conditions of installation via the strength member 110 and jacket 112 (which is loose between the anchoring locations). This provides significantly improved monitoring of shear and tension deformations (See FIGS. 4 and 5) within the installation medium 200. It also provides crack detection with a spatial resolution akin to that of the sensing system being used.

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A fiber optic cable system configured for distributed sensing in a brittle installation medium, the fiber optic cable system comprising:
a fiber optic cable comprising:
a fiber core configured to carry an optical signal;
a buffer layer surrounding the fiber core;
a strength member surrounding the buffer layer and the fiber core; and
a jacket surrounding the fiber core, the buffer layer, and the strength member,
the fiber core, the buffer layer, the strength member, and the jacket being secured together at a plurality of anchoring locations along the fiber optic cable so that the fiber core, the buffer layer, the strength member, and the jacket are bonded together at the plurality of anchoring locations and un-bonded from each other between the plurality of anchoring locations.

2. The fiber optic cable system of claim 1, further comprising:
a plurality of anchors positioned at the plurality of anchoring locations, each of the plurality of anchors comprising:
structure configured to engage the brittle installation medium, the structure defining an infiltration space such that the brittle installation medium infiltrates the infiltration space when the brittle installation medium is in an at least semi-liquid state so that the structure is fixed in the brittle installation medium when the brittle installation medium is in a solid state.

3. The fiber optic cable system of claim 1, the jacket being compressed over the strength member at the plurality of anchoring locations.

4. The fiber optic cable system of claim 1, the plurality of anchoring locations being spaced an interval length apart from each other.

5. The fiber optic cable system of claim 2, the structure including a plurality of radially-protruding ribs, the infiltration space being between the plurality of radially-protruding ribs.

6. The fiber optic cable system of claim 2, the plurality of anchors each having opposing first and second ends, the plurality of radially-protruding ribs merging together at the opposing first and second ends.

7. The fiber optic cable system of claim 2, the plurality of anchors being at least one of secured to the fiber optic cable via epoxy resin, secured to the fiber optic cable via a clamping device, or integrated into the fiber optic cable.

8. A method of fabricating a fiber optic cable, the method comprising a step of:
securing a fiber core, a, strength member, and a jacket together at a plurality of anchoring locations spaced an interval length apart from each other so that the fiber core, the strength member, and the jacket are bonded together at the plurality of anchoring locations and un-bonded from each other between the plurality of anchoring locations.

9. The method of claim 8, the securing step including a step of melting the jacket at a plurality of anchoring locations.

10. The method of claim 8, the securing step including a step of compressing the jacket around the fiber core at the plurality of anchoring locations.

11. The method of claim 9, the securing step including a step of cooling the jacket.

12. The method of claim 8, the securing step including a step of coupling the fiber core and the jacket at the plurality of anchoring locations.

13. The method of claim 8, the securing step including a step of crimping the jacket at the plurality of anchoring locations.

14. An anchor for a fiber optic cable of a fiber optic cable system configured for distributed sensing in a brittle installation medium, the anchor comprising:
structure configured to engage the brittle installation medium, the structure defining an infiltration space such that the brittle installation medium infiltrates the infiltration space when the brittle installation medium is in an at least semi-liquid state so that the structure is fixed in the brittle installation medium when the brittle installation medium is in a solid state.

15. The anchor of claim 14, the structure including a plurality of radially-protruding ribs, the infiltration space being between the plurality of radially-protruding ribs.

16. The anchor of claim 15, the anchor having opposing first and second ends, the plurality of radially-protruding ribs merging together at the opposing first and second ends.

17. The anchor of claim 14, the structure defining a plurality of fastener apertures configured to receive cinching fasteners therethrough for securing the anchor to the fiber optic cable.

18. The anchor of claim 14, the structure further defining a plurality of fastener guides configured to receive portions of the cinching fasteners.

19. The anchor of claim 14, the anchor being secured to the fiber optic cable via epoxy resin or a clamping device or being integrated into the fiber optic cable.

20. The anchor of claim 14, the anchor having opposing first and second ends, the structure including four radially-protruding ribs merging together at the opposing first and second ends, the four radially-protruding ribs defining a plurality of fastener apertures configured to receive cinching fasteners therethrough and a plurality of fastener guides configured to receive portions of the cinching fasteners for securing the anchor to the fiber optic cable, the infiltration space being between the plurality of radially-protruding ribs.
